(19) United States
(12) Patent Application Publication
(10) Pub. No.: US 2021/0232901 A1
(43) Pub. Date: Jul. 29, 2021

(54) ELECTROPHYSIOLOGICAL SIGNAL PROCESSING METHOD, CORRESPONDING SYSTEM, VEHICLE AND COMPUTER PROGRAM PRODUCT

(71) Applicant: STMicroelectronics S.r.l., Agrate Brianza (IT)

(72) Inventors: Francesco Rundo, Gravina di Catania (IT); Sabrina Conoci, Tremestieri Etneo (IT); Concetto Spampinato, Catania (IT)

(21) Appl. No.: 17/156,052

(22) Filed: Jan. 22, 2021

(30) Foreign Application Priority Data
Jan. 24, 2020 (IT) 102020000001393

(51) Int. Cl. G06N 3/063; G06N 3/04

(52) U.S. Cl. CPC G06N 3/063; B62D 1/046; G06N 3/0454

(57) ABSTRACT

An embodiment method includes segmenting at least one electrophysiological signal and producing a set of sampled waveforms, applying artificial neural network processing to the set of sampled waveforms and a set of randomly generated noise samples and producing at least one altered data pattern, the altered data pattern comprising the set of filtered waveforms altered as a function of the randomly generated noise samples, providing calibration data comprising expected waveforms for filtered waveforms in the set of filtered waveforms, applying classifier processing to the produced at least one altered data pattern to detect a degree of resemblance between the produced at least one altered data pattern and the calibration data patterns, the classifier processing producing classification signals having values above or below at least one threshold value as a function of the detected degree of resemblance, and triggering a user circuit as a function of the classification signal.

### CROSS-REFERENCE TO RELATED APPLICATIONS

This application claims the benefit of Italian Application No. 102020000001393, filed on Jan. 24, 2020, which application is hereby incorporated herein by reference.

### TECHNICAL FIELD

The description relates to a method and system for electrophysiological signal processing such as, for instance, PhotoPlethysmoGraphy (PPG) signals.

One or more embodiments may be useful in obtaining information from a driver of a vehicle with a view to possibly generating alert signals and/or activating safety procedures (e.g. taking over control of the vehicle), for instance within the framework of an Advanced Driver-Assistance System (ADAS).

### BACKGROUND

Drowsiness of a vehicle driver (before and during driving) may adversely affect driving safety. Driver's drowsiness may cause serious road traffic accidents involving vehicles. The possibility to detect an attention state of a driver may facilitate evaluation of his/her fitness to drive a vehicle, facilitating to prevent road accidents.

Specifically, it may be interesting for improving driving safety to be able to perform operations of:
driver age detection, e.g., to facilitate insurance risk classification,
driver eyes motion tracking, so as to discriminate whether eyes are closed due to a blink or for a relatively long time (e.g., more 10 seconds).

Vision systems, e.g., based on a camera installed within the vehicle, may be used to obtain such operations. Yet, such systems may fail in specific conditions, for instance due to poor lighting of the passenger compartment (night driving) or due to the presence of impediments, for instance such as due to sunglasses being worn by the driver. Installing and integrating a vision system in the vehicle may come at an increased costs and complexity.

PhotoPlethysmoGraphy (PPG) is a simple and low-cost optical technique that can be used to detect blood volume changes in the microvascular bed of tissue. It is often used non-invasively to make measurements at the skin surface.

A PPG waveform comprises a pulsatile (‘AC’) physiological waveform attributed to cardiac synchronous changes in the blood volume with each heartbeat, and is superimposed on a slowly varying (‘DC’) baseline with various lower frequency components attributed to respiration, thermoregulation, skin tissues, etc.

For each cardiac cycle the heart pumps blood to the periphery. Even though this pressure pulse is somewhat damped by the time it reaches the skin, it is enough to distend the arteries and arterioles in the subcutaneous tissue. If a light reflex/transmit detector device is attached over the skin, a pressure pulse can also be seen from the venous plexus, as a small secondary peak. The change in volume caused by the pressure pulse is detected by illuminating the skin with the light from a light-emitting diode (LED) and then measuring the amount of light either transmitted or reflected to a photodiode. Each cardiac cycle appears as a peak.

Because blood flow to the skin can be modulated by multiple other physiological systems, the PPG can also be used to monitor breathing, hypovolemia, and circulatory conditions as well as for subjective analysis. Additionally, the shape of the PPG waveform differs from subject to subject, and varies with the location and manner in which the pulse oximeter is attached.

As mentioned, an extensive activity is carried on and several approaches are proposed in literature, for instance in:
[1] Jeyhani, Vala et al.: “Comparison of HRV parameters derived from photoplethysmography and electrocardiography signals”, 2015, 37th Annual International Conference of the IEEE Engineering in Medicine and Biology Society (EMBC). 2015, pp. 5952-5955, discussing HRV analysis applied on beat-to-beat intervals obtained from ECG and PPG and discussing some important HRV parameters calculated from PPG-HRV and ECG-HRV, wherein maximum of PPG and its second derivative were considered as two methods for obtaining the beat-to-beat signals from PPG and the results were compared with those achieved from ECG-HRV;
[2] M. Bolanos, et al.: “Comparison of Heart Rate Variability Signal Features Derived from Electrocardiography and Photoplethysmography in Healthy Individuals”, 2006, International Conference of the IEEE Engineering in Medicine and Biology Society, New York, N.Y., 2006, pp. 4289-4294, discusses heart rate variability (HRV) signal used as a noninvasive marker in monitoring the physiological state of an individual and a PDA-based system developed to simultaneously record ECG and PPG signals to facilitate accurately controlled sampling and recording durations, wherein a comparison between different features of the HRV signals derived from both methods was performed to test the validity of using PPG signals in HRV analysis, using autoregressive (AR) modeling, Poincare’ plots, cross correlation, standard deviation, arithmetic mean, skewness, kurtosis, and approximate entropy (ApEn) to derive and compare different measures from both ECG and PPG signals, providing potential support for the idea of using PPGs instead of ECGs in HRV signal derivation and analysis in ambulatory cardiac monitoring of healthy individuals;
[3] Vicente, Jose et al.: “Detection of driver's drowsiness by means of HRV analysis”, 2011, Computing in Cardiology (2011): p. 89-92, discusses driver's drowsiness detection based on biological and vehicle signals being studied in preventive car safety based on Autonomous Nervous System (ANS) activity which can be measured non-invasively from the Heart Rate Variability (HRV) signal obtained from surface ECG, presenting alterations during stress, extreme fatigue and drowsiness episodes in the hypothesis is that these alterations manifest on HRV, wherein an on-line detector of driver's drowsiness based on HRV analysis is developed, wherein two databases have been analyzed: one of driving simulation in which subjects were sleep deprived, and the other of real situation with no sleep deprivation;
[4] G. Ryu et al., “Flexible and Printed PPG Sensors for Estimation of Drowsiness” in IEEE Transactions on Electron Devices, vol. 65, no. 7, pp. 2997-3004, July 2018, discusses printed flexible optoelectronic sensors composed of red organic light-emitting diodes (OLEDs) and organic photodiodes (OPDs) for detection of various biological signals in a photoplethysmogram (PPG) device, wherein PPG signals were successfully detected using the developed flexible PPG sensor and the conventional driving circuit, wherein subject drowsiness was estimated from heart rate variability, extracted from the PPG signals, using machine learning algorithms;
[5] Sari, Nila Novita et al.: “A two-stage intelligent model to extract features from PPG for drowsiness detection”, 2016, International Conference on System Science and Engineering (ICSSE) (2016), p. 1-2, discusses a two-stage intelligent model that combined the wavelet packet transform (WPT) and functional-link-based fuzzy neural network (FLFNN) to access drowsy level for early detection of drowsiness employing a sensor device that detects drowsy status at an early stage;
[6] Kurian, Deepu et al.: “Drowsiness Detection Using Photoplethysmography Signal”, 2014, Fourth International Conference on Advances in Computing and Communications (2014), P. 73-76, discusses an approach to detect drowsiness by using photoplethysmography signals, easily acquirable with non-invasive techniques, wherein Autonomous Nervous System (ANS) activity can be measured non-invasively from the Pulse Rate Variability (PRV) signal obtained from photoplethysmography signal (PPG), that comprises alterations during, relaxation, extreme fatigue and drowsiness episodes, developing an on-line detector of drowsiness based on PRV analysis, wherein the databases have been collected with the aid of an external observer who decides upon each minute of the recordings as drowsy or awake, and constitutes our data base;
[7] Lee, B.-G. et al.: “Real-time physiological and vision monitoring of vehicle driver for non-intrusive drowsiness detection”, IET Communications 5, 2011, p. 2461-2469, discusses an approach to detect driver's drowsiness by applying two distinct methods in computer vision and image processing, wherein the objective is to combine both methods under one single profile instead of relied solely on a detection method to enhance the driver's drowsiness detection resolution, therefore a non-intrusive drowsy-monitoring system is developed to alert the driver if driver falls into low arousal state, wherein photoplethysmography (PPG) is analyzed for its changes in signals waveform from awake to drowsy state, meanwhile eyes pattern or motion in image processing is addressed to detect driver fatigue, wherein genetic algorithm with template-matching approach is designed to detect eye region and estimate the drowsiness in different metric standard based on eyes behavior, wherein PPG drowsy signals are integrated with eyes motion to derive the final probability model for delivering valid and reliable drowsiness detection system,
[8] S. b. Usman, M. A. b. M. Ali, M. M. B. I. Reaz and K. Chellapan, “Second derivative of photoplethysmogram in estimating vascular aging among diabetic patients,” 2009 International Conference for Technical Postgraduates (TECHPOS), Kuala Lumpur, 2009, pp. 1-3.

As mentioned, various solutions proposed in the literature may be exposed to one or more of the following drawbacks:
vascular aging may be computed based on a combination of PPG together with ECG pulse transit time (PTT);
vascular aging depends of health information of the subject,
existing algorithms may present high computational costs and low prediction/accuracy capabilities.

Existing solutions hence lack a fine detection of a change in the person/driver physiology, especially while employing relatively cheap and low complexity components.

### SUMMARY

Despite the extensive activity in the area, improved solutions facilitating, for instance, identifying a drowsy state of a vehicle driver are desirable.

According to one or more embodiments, such an object can be achieved by means of a method having the features set forth in the claims that follow.

A bio-inspired method of processing electrophysiological signals comprising applying artificial neural network processing may be exemplary of such a method.

One or more embodiments may relate to a corresponding system.

One or more embodiments may relate to a corresponding vehicle, such as, for instance, a motor vehicle equipped with such a system.

One or more embodiments may comprise a computer program product loadable in the memory of at least one processing circuit (e.g., a computer) and comprising software code portions for executing the steps of the method when the product is run on at least one processing circuit. As used herein, reference to such a computer program product is understood as being equivalent to reference to computer-readable medium containing instructions for controlling the processing system in order to co-ordinate implementation of the method according to one or more embodiments. Reference to “at least one computer” is intended to highlight the possibility for one or more embodiments to be implemented in modular and/or distributed form.

The claims are an integral part of the technical teaching provided herein in respect of the invention.

One or more embodiments may facilitate providing time-continuous attention level monitoring for a vehicle driver on-board the vehicle, e.g. increasing road safety.

One or more embodiments comprise advanced time-based efficient and robust near real-time detection of continuous driver's level of attention, by using sampled PPG signal of the same driver.

One or more embodiments may comprise PPG detectors performing sampling of PPG time series of a car-driver (e.g., from solely one hand placed on the car steering, hence advantageously employing a single detection point).

One or more embodiments thus facilitate obtaining information (data, physical quantities) from the living human or animal body e.g. in support the diagnostic activity of a human in medical and veterinary activities or for other possible uses. Obtaining information on the behavior and/or the reaction of drivers and passengers in the automotive field is exemplary of one such possible use.

One or more embodiments may comprise a vehicle equipped with such PPG detectors and with such ADAS system configured to process signals detected by the PPG detectors.

One or more embodiments may facilitate continuous driver drowsiness detection/monitoring without the employ of frequency domain computations as well as without lengthy data-buffering.

One or more embodiments may use solely electrophysiological signal, e.g. PPG signal, samples for providing drowsiness detection with approximately 96% of accuracy, advantageously eliminating the presence of multiple signal types detection systems (e.g., Vision).

One or more embodiments may facilitate overcoming vision system drawbacks, providing the possibility to detect an attention level and/or an age of a driver of a vehicle even when obstacles may be placed in a field of view of a vision system, for instance if the driver wears sunglasses or has closed eyes.

One or more embodiments may comprise an ad-hoc high-speed machine learning pipeline configured to process such electrophysiological signal features/dynamics from filtering, facilitating to provide drowsy/wakeful classification of driver's state.

One or more embodiments may facilitate reaching high accuracy rate and sensitivity/specificity ratio.

One or more embodiments may advantageously be implemented over embedded electronic devices.

One or more embodiments may advantageously avoid using sensitive personal healthcare information or data.

The claims are an integral part of the technical teaching provided herein with reference to the embodiments.

### BRIEF DESCRIPTION OF THE DRAWINGS

One or more embodiments will now be described, by way of non-limiting example only, with reference to the annexed Figures, wherein:

FIG. 1 is a diagram exemplary of a PhotoPlethysmoGraphy (PPG) signal;

FIG. 2 is exemplary of possible operation of PPG sensors as disclosed herein;

FIG. 3 is a diagram exemplary of a method as per the present disclosure,

FIG. 4 comprising portions a), b) and c) represent diagrams of a possible set of filtered waveforms as per the present disclosure,

FIG. 5 is a diagram exemplary of a portion of the diagram of FIG. 3,

FIGS. 6 and 7 are exemplary diagrams of a non-linear cellular neural network as per the present disclosure,

FIG. 8 is an exemplary diagram of a convolutional neural network as per the present disclosure.

[symbol]

_{GAN}(G,D)= [symbol] _{y˜p|}_{_{y}}[log D(Y)]

G*=argmin_{G} argmax_{D} [symbol] _{GAN}

[symbol]

_{GAN}(G,D)= [symbol] _{x,y˜p|}_{_{x,y}}[log D(X,Y)]+ [symbol] _{x˜p|}_{ₓ}_{,z˜p|}_{_{z}}[log(1−D(X,G(X,Z)))]

wherein

p|x,y and p|z are respective probability values.

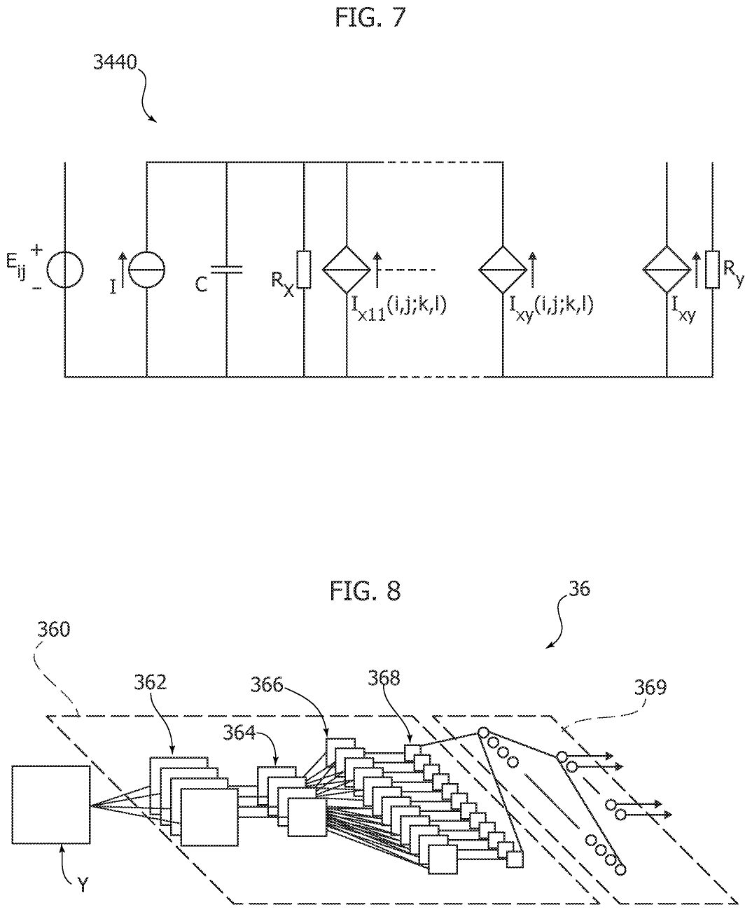

In one or more embodiments, the second generator stage 344 may be implemented using non-linear cellular neural network (briefly, NL-CNN) technology, thus offering high-speed computing speeds. Specifically, one or more embodiments may exploit a NL-CNN computation paradigm as exemplified in FIGS. 6 and 7 to generate altered datasets Y as a function of the input segmented PPG waveforms WF and data patterns P produced by the first ANN 342 starting from generated 340 random noise values Z.

Non-Linear Cellular Neural Networks, briefly NL-CNNs, comprise arrays of nonlinear and simple computing elements or cells, comprising local interactions between cells. A CNN paradigm is thus well suited to describe locally interconnected simple dynamical systems showing a lattice-like structure.

Such (NL-)CNNs arrangements suitable for use in one or more embodiments are discussed, for instance, in documents:
L. O. Chua, L. Yang: “Cellular neural networks: theory”, IEEE Transactions on Circuits and Systems 1988 Volume: 35, Issue: 10 Pages: 1257-1272;
L. O. Chua, L. Yang: “Cellular neural networks: applications”, IEEE Transactions on Circuits and Systems 1988 Volume: 35, Issue: 10 Pages: 1273-1290
A. Zarandy, A. Stoffels, T. Roska, L. O. Chua: “Morphological operators on the CNN Universal Machine”, Fourth IEEE International Workshop on Cellular Neural Networks and their Applications Proceedings (CNNA-96), 1996 Pages: 151-156.

NL-CNNs may be used for various types of applications such as image and signal processing, bio-inspired system modelling, or high-speed resolution of partial differential equations (PDEs).

One or more embodiments may adopt an analog implementation of a cell 3440 of the NL-CNN 344 as exemplified in FIG. 7, which may correspond to the analytic and circuit model of a NL-CNN cell 3440 which can be expressed as:

$$\frac{\partial {x}_{j}}{\partial t} = g [ {x}_{j} ( t ) ] + {\sum }_{γ ∈ {N}_{r} ( j )} {{ϑ}_{j}}_{} ( {x}_{j} {\vert}_{( t - τ , t ]} , {y}_{γ} {\vert}_{( t - τ , t ]} ; {p}_{j}^{A} ) + {\sum }_{γ ∈ {N}_{r} ( j )} {{φ}_{j}}_{} ( {x}_{j} {{\vert}_{( t - τ , t ]} , {u}_{γ} \vert}_{( t - τ , t ]} ; {p}_{j}^{B} ) + {\sum }_{γ ∈ {N}_{r} ( j )} {{ρ}_{j}}_{} ( {x}_{j} {{\vert}_{( t - τ , t ]} , {x}_{γ} \vert}_{( t - τ , t ]} ; {p}_{j}^{C} ) + {I}_{j} ( t ) \\ {y}_{j} ( t ) = 𝔉 ( {x}_{j} {\vert}_{( t - τ , t ]} ) \\ C \frac{{dx}_{ij} ( t )}{dt} = - \frac{1}{{R}_{x}} {x}_{ij} + {\sum }_{C ( k , l ) ∈ {N}_{i} ( i , j )} A ( i , j ; k , l ) {y}_{kl} ( t ) + {\sum }_{C ( k , l ) ∈ {N}_{i} ( i , j )} B ( i , j ; k , l ) {u}_{kl} ( t ) + {\sum }_{C ( k , l ) ∈ {N}_{i} ( i , j )} C ( i , j ; k , l ) {x}_{kl} ( t ) + {\sum }_{C ( k , l ) ∈ {N}_{i} ( i , j )} D ( i , j ; k , l ) ( {y}_{ij} ( t ) , {y}_{kl} ( t ) ) + I ( 1 \leq i \leq M , 1 \leq j \leq N ) \\ {y}_{ij} ( t ) = \frac{1}{2} ( \vert {x}_{ij} ( t ) + 1 \vert - \vert {x}_{ij} ( t ) - 1 \vert ) \qquad$$

The dynamics of a NL-CNNs cell C(i,j) as exemplified in FIG. 6 is described by the equations reported above which the state of the cell is represented by generator current (Iₓ₁₁) while input and output of the neighborhood coupled cells is represented by the voltages “vᵤm” and “v_{ykl}” respectively. The neighborhood of single cell C(i,j) is mathematically represented by Nr(i,j) while the type of cell-coupling is defined by the elements of the so-called cloning matrix templates (or image grids) A(I,j;k,l), B(I,j;k,l), C(I,j;k,l) as well as by the bias I.

The output voltage of single cell “v_{yij}(t)” is defined by PieceWise Linear (PWL) remapping of the state of the cell C(i,j). A VLSI implementation of NL-CNNs involving so-called State-Controlled CNNs (SC-CNNs), where a C(I,j;k,l) matrix template (or image grid) is added allows high-speed computation of single cell dynamic.

FIG. 8 is a diagram exemplary of classifier processing 36, specifically image processing wherein the input data has size 32×2.

In one or more embodiments as exemplified in FIG. 8, as mentioned, the classification layer may use convolutional neural network, CNN, processing, which may comprise:
a first data processing sub-stage 360, comprising:

a) an input layer, configured to receive as input data the set of produced altered dataset(s) Y (and/or calibration data X during training 35) as entries of image grid (or matrix) arranged as grayscale images having pixel values equal to values of the altered dataset Y;

b) at least one convolutional layer 362, 366 comprising at least one kernel, for instance having a kernel size of 3-by-3, configured to be convoluted with the input data Y; and comprising a number of perceptrons that connect to the same region of the input, wherein such a number of perceptron may define the dimension of the feature maps generated by the CNNs;
at least one nonlinear activation function, coupled to the at least one convolutional layer 362, for instance comprising a rectified linear unit (briefly, ReLU) function;
at least one max pooling layer 364, 368, coupled to the at least one nonlinear activation function, configured to apply down-sampling operation that reduces the spatial size of the feature map and to remove redundant spatial information, facilitating to increase the number of filters in deeper convolutional layers without increasing the required amount of computation per layer, wherein the max pooling layer returns the maximum values of rectangular regions of feature input image coming from previous layer;
a second data processing sub-stage 369, comprising at least one fully Connected Layer, wherein all perceptrons connect to all the perceptrons in the preceding layer, which is configured to combine all the features learned by the previous layers across the image to identify the larger patterns; hence a last fully connected layer combines the features to classify the images.

In one or more embodiments the at least one fully Connected Layer may comprise a softmax Layer, comprising perceptrons configured to apply a so-called softmax activation function to normalize the output of the fully connected layer. For instance, the output of the softmax layer consists of positive numbers that sum to one, which can then be used as classification probabilities by the classification layer.

In one or more embodiments, the final layer of the classifier 36 may be a classification layer, wherein probabilities returned by the softmax activation function for each input to assign the input to one of the mutually exclusive classes and compute the loss and performance indexes.

As repeatedly noted in the foregoing, PPG processing apparatus as discussed herein lends itself to be used in areas other than the medical field, e.g. in the automotive field in order to gain useful information on the behavior and/or the reaction of drivers and passengers in various situations which may occur in a motor vehicle.

One or more embodiments have been tested using several PPG measurements collected from drivers in known conditions, e.g. for instance data from more than 70 patients with different ages, sex, and so on, under Physiologists directive. Specifically, a training database comprised PPG signals collected with drivers having different ages both in a Drowsy state and with closed eyes and as well as in a Wakeful state with open eyes,

The performed test has proved that the method has a validation Accuracy of 96-97% as eyes tracker or driver age detector.

In one or more embodiments, calibration or training 35 may be performed on Intel i9 12 Cores 32 Gbyte-NVIDIA RTX Tit. 2080 GPU processing circuits.

As exemplified herein, a method (for instance, 30) of processing at least one electrophysiological signal (for instance, S) may include operations of:

segmenting (for instance, 32) the at least one electrophysiological signal and producing a set of sampled waveforms (for instance, WF) as a result,

producing a set of randomly generated noise samples (for instance, Z),

applying artificial neural network processing (for instance, 34) to the set of sampled waveforms and to the set of randomly generated noise samples and producing at least one altered data pattern (for instance, Y) as a result, the altered data pattern comprising the set of filtered waveforms altered as a function of the randomly generated noise samples,

providing (for instance, 35) calibration data (for instance, X) comprising expected waveforms for filtered waveforms in the set of filtered waveforms,

applying classifier processing (for instance, 36) to the produced at least one altered data pattern to detect a degree of resemblance between the produced at least one altered data pattern and the calibration data patterns, wherein the classifier processing produces classification signals (for instance, T) having values above or below at least one threshold value as a function of the detected degree of resemblance,

triggering a user circuit (for instance, A) as a function of the classification signal (for instance, T).

As exemplified herein, the at least one electrophysiological signal may comprise at least one photopletysmography, PPG, signal.

As exemplified herein, the at least one electrophysiological signal may be collected from a driver (for instance, D) of a vehicle (for instance, V), and the user circuit triggered as a function of the classification signal may comprise a user circuit on board the vehicle.

As exemplified herein, the at least one electrophysiological signal is collected from the driver (D) of the vehicle (V) via a PPG sensor (20) placed on board a steering wheel (SW) of the vehicle (V).

As exemplified herein, the operation of applying artificial neural network processing (34) comprises:
applying a first artificial neural network processing (for instance, 342) to the set of randomly generated noise signals and providing a first set of generated data patterns (for instance, P),
applying a second artificial neural network processing (for instance, 344) to the first set of generated data patterns (for instance, P) and to the set of filtered waveforms (for instance, WF) and providing the at least one altered data pattern (for instance, Y),

wherein the second artificial neural network (for instance, 344) is configured to provide the at least one altered data pattern as a result of altering the set of filtered waveforms as a function of the first set of generated data patterns.

As exemplified herein, the operation of applying a second artificial neural network processing comprises using, applying, a non-linear cellular neural network, NL-CNN, circuit.

As exemplified herein:

the operation of applying classifier processing (for instance, 36) comprises using convolutional neural network, CNN, processing,

the operation of applying a first artificial neural network processing (for instance, 342) comprises using an inverse convolutional neural network processing.

As exemplified herein, the method may comprise at least one of:
using the calibration data (for instance, 35) and the classification signal to configure the classifier processing stage to reduce occurrences of the classification signal having a value indicative of a detected degree of resemblance,
using the classification signal (for instance, T) to configure the artificial neural network processing stage (for instance, 34) to increase occurrences of the classification signal having a value indicative of a detected degree of resemblance.

As exemplified herein, an electrophysiological signal processing system (for instance, 100) configured to be coupled to at least one electrophysiological signal sensor (for instance, 20) collecting from a human (for instance, D) at least one electrophysiological signal (for instance, S, WF), may comprise processing circuitry configured to receive the electrophysiological signal (for instance, S) and to perform operations of:

segmenting (for instance, 32) the at least one electrophysiological signal and producing a set of sampled waveforms (for instance, WF) as a result,

producing a set of randomly generated noise samples (for instance, Z),

applying artificial neural network processing (for instance, 34) to the set of sampled waveform and to the set of randomly generated noise samples and producing at least one altered data pattern (for instance, Y) as a result, the altered data pattern comprising the set of filtered waveforms altered as a function of the randomly generated noise samples,

providing (for instance, 35) calibration data (for instance, X) comprising expected waveforms for filtered waveforms in the set of filtered waveforms,

applying classifier processing (for instance, 36) to the produced at least one altered data pattern to detect a degree of resemblance between the produced at least one altered data pattern and the calibration data patterns, wherein the classifier processing produces classification signals (for instance, T) having values above or below at least one threshold value as a function of the detected degree of resemblance,

triggering a user circuit (for instance, A) as a function of the classification signal.

As exemplified herein, a vehicle (for instance, V may be) equipped with the system as exemplified herein in combination with at least one electrophysiological signal sensor (for instance, 20) configured to collect at least one electrophysiological signal (S, WF), preferably a photopletismography, PPG, sensor configured to collect a PPG signal.

As exemplified herein, the vehicle (V) may comprise at least one driver assistance device (for instance, A) configured to be triggered as a function of the classification signal (for instance, T).

As exemplified herein, a computer program product may be loadable into the memory of at least one processing circuit (for instance, 100) and comprising software code portions for executing the steps of the method (for instance, 30) as exemplified herein when the product is run on at least one processing circuit.

It will be otherwise understood that the various individual implementing options exemplified throughout the figures accompanying this description are not necessarily intended to be adopted in the same combinations exemplified in the figures. One or more embodiments may thus adopt these (otherwise non-mandatory) options individually and/or in different combinations with respect to the combination exemplified in the accompanying figures.

Without prejudice to the underlying principles, the details and embodiments may vary, even significantly, with respect to what has been described by way of example only, without departing from the extent of protection. The extent of protection is defined by the annexed claims.

### DETAILED DESCRIPTION OF ILLUSTRATIVE EMBODIMENTS

In the ensuing description, one or more specific details are illustrated, aimed at providing an in-depth understanding of examples of embodiments of this description. The embodiments may be obtained without one or more of the specific details, or with other methods, components, materials, etc. In other cases, known structures, materials, or operations are not illustrated or described in detail so that certain aspects of embodiments will not be obscured.

Reference to “an embodiment” or “one embodiment” in the framework of the present description is intended to indicate that a particular configuration, structure, or characteristic described in relation to the embodiment is comprised in at least one embodiment. Hence, phrases such as “in an embodiment” or “in one embodiment” that may be present in one or more points of the present description do not necessarily refer to one and the same embodiment.

Moreover, particular conformations, structures, or characteristics may be combined in any adequate way in one or more embodiments.

The references used herein are provided merely for convenience and hence do not define the extent of protection or the scope of the embodiments.

The drawings are in simplified form and are not to precise scale. For the sake of simplicity, directional (up/down, etc.) or motional (forward/back, etc.) terms may be used with respect to the drawings. The term “couple” and similar terms do not necessarily denote direct and immediate connections, but also include connections through intermediate elements or devices.

For the sake of simplicity, principles underlying the invention are discussed in the following mainly in relation to the processing of a PhotoPletysmoGraphy (PPG) signal. Such an electrophysiological signal type is purely exemplary, being otherwise understood that other types of electrophysiological signals may be processed in one or more embodiments, e.g. ElectroCardioGram (ECG) signals, ElectroEncephaloGram (EEG) signals, etc.

In one or more embodiments, a PPG signal may be simpler to employ as electrophysiological to process according to a method as disclosed herein, as it may be easier to sample in an automotive environment with respect to an ECG/EEG signal, due to a reduced invasiveness of the hardware in the limited volume of a vehicle.

FIG. 1 is a diagram exemplary of a PhotoPlethysmoGraphy (PPG) signal.

As exemplified in FIG. 1, a typical PhotoPlethysmoGraphy (briefly PPG) signal/waveform comprises:
a systolic peak SP at a peak value x,
a dicrotic notch DN,
a distolic peak DP at a value y.

A width W of the pulse may also be defined at a given value of the PPG value.

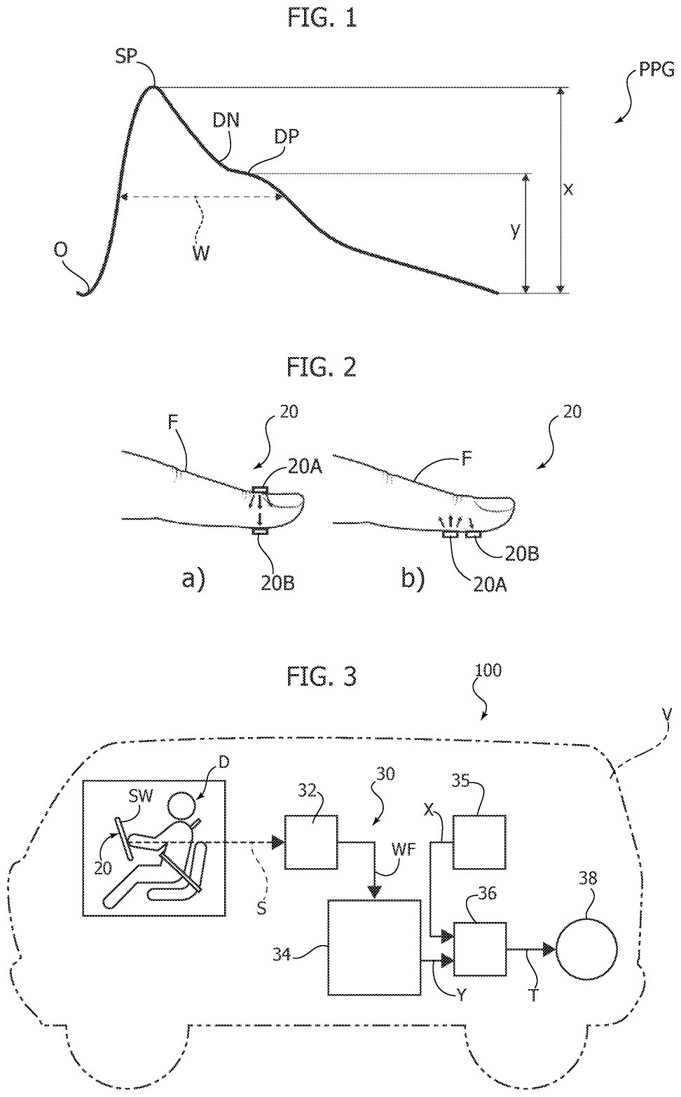

PPG signals can be detected by using PPG sensors/devices (e.g., sensor 20 in FIG. 2) comprising LED emitters 20A operating at specific wavelengths (usually infrared at 940 nm) and receivers 20B comprising silicon photomultipliers or SiPMs (see e.g. M. Mazzillo, et al.: “Silicon Photomultiplier technology at STMicroelectronics”, IEEE Trans. Nucl. Sci, vol. 56, no. 4, pp. 2434-2442, 2009). For instance, the PPG signal S may be sampled by vehicle probes placed on the steering wheel SW.

These SiPMs may have a total area of 4.0×4.5 mm2 and 4871 square microcells with 60 μm pitch (1 μm=1 micron=10⁻⁶ m). These devices 20B have a geometrical fill factor of 67.4% and are packaged in a surface mount housing (SMD) with 5.1×5.1 mm² total area (see e.g. M. Mazzillo, et al., cited above or M. Mazzillo, et al.: “Electro-optical performances of p-on-n and n-on-p silicon photomultipliers”, IEEE Trans. Electron Devices, vol. 59, no. 12, pp. 3419-3425, 2012).

A Pixelteq dichroic bandpass filter with a pass band centered at 542 nm with a Full Width at Half Maximum (FWHM) of 70 nm (1 nm=10⁻⁹ m) and an optical transmission higher than 90% in the pass band range can be glued on the SMD package by using a Loctite® 352™ adhesive. With the dichroic filter at 3V-OV the SiPM has a maximum detection efficiency of about 29.4% at 565 nm and a PDE of about 27.4% at 540 nm (central wavelength in the filter pass band −1 nm=10⁻⁹ m). It was noted that the dichroic filter can reduce in excess of 60% the absorption of environmental light in the linear operation range of the detector operating in Geiger mode above its breakdown voltage (˜27V). OSRAM LT M673 LEDs in SMD package emitting at 529 nm (1 nm=10⁻⁹ m) and based on InGaN technology have been used as optical light sources in exemplary embodiments. These LEDs 20B have an area of 2.3×1.5 mm2, viewing angle of 120°, spectral bandwidth of 33 nm (1 nm=10⁻⁹ m) and typical power emission of a few mW in the standard operation range.

Use of PPG probes 20 comprising Silicon PhotoMultiplier (SiPM) detectors 20B may provide advantages in terms of single-photon sensitivity and high internal gain for relatively low reverse bias.

It was observed (see e.g. D. Agrò, et al.: “PPG embedded system for blood pressure monitoring,” in AEIT Annual Conference—From Research to Industry: The Need for a More Effective Technology Transfer (AEIT), Trieste, 2014), that Silicon PhotoMultipliers (SiPMs) can provide advantages in PPG detecting systems 20 in terms of higher AC-to-DC ratio in PPG pulse waveform, high repeatability and immunity to motion artifacts and ambient interferences. One or more embodiments as discussed herein may sense PPG signals by using SiPMs (as available with companies of the ST group) as optical probe sensors, adapted to be used in conjunction with hardware and software components in providing a signal processing pipeline.

FIG. 2 is exemplary of possible operation of PPG sensors 20 when activated, for instance by the contact of a finger on the sensor surface.

Light emitted by the LEDs 20A may be absorbed by the skin (DC component) and the arteries, specifically, by oxygenated (and partly by de-oxygenated) hemoglobin (AC component). Residual propagated/reflected (back-scattered) light may be a function (proportional-differential) of the amount of light absorbed by blood hemoglobin in the various heart phases (systolic, diastolic, dicrotic, etc.). A SiPM photomultiplier 20B may thus detect the presence of photons in the propagated/reflected light by transducing an electrical signal that can be sampled by an e.g. 24-bit ADC thus providing PPG signal as discussed previously.

Such PPG sensors 20 may be applied on a steering device of a vehicle, in various arrangements as exemplified in FIG. 2, for instance:
a first “transmission” arrangement, as exemplified in portion a) of FIG. 2, wherein radiation from the LED 20A may be propagating through the body (e.g. the body of a patient being clinically investigated or a driver), for instance through a fingertip F, and
a second “reflection” arrangement as exemplified in portion b) in FIG. 2, wherein radiation from the LED 20A may be reflected (back-scattered) from the body F, facilitating relaxation of the requirements for possible positioning of the PPG sensors/detectors 20 with respect to the body.

In one or more embodiments, the PPG detector device 20 may comprise PPG probe circuitry 20A, 20B comprising a PPG probe section and a printed circuit board (PCB) configured for interfacing the probe sections with an acquisition and processing circuit.

As exemplified in FIG. 3, one or more embodiments of a system 100 of electrophysiological signal processing which may be used for instance on-board a vehicle V, the system comprising:
at least one PPG detector device 20, for instance mounted onto the steering wheel SW of a vehicle V, configured to detect PPG signals S of a driver D,
processing circuitry 30 configured to receive the collected PPG signals S and to apply an electrophysiological signal processing pipeline 30 thereto, as discussed in the following, producing at least one signal T to be provided to user circuits,
one or more interface circuits 38, for instance a display unit, a sound and/or light generator, and so on, and/or an ADAS stage, configured to receive the produced classification signal T and to trigger activation/deactivation of the vehicle V or on-screen display of alert messages to the driver D of the vehicle V.

As mentioned, the results produced by the system 100 can possibly be presented on a display unit 38 to an operator, e.g. a medical practitioner, with the capability of supporting his activity, e.g. for diagnostic purposes.

In one or more embodiments, the interface stage 38 of the system 10 may comprise an Advanced Driver Assistance System (ADAS) configured to receive the indicator T and to use it in assisting the car driving operations, for instance providing an alert to the driver D of the vehicle V as a result of the indicator T being below or above a certain threshold and/or taking control over the vehicle in case a drowsy driver state is detected, potentially improving driving safety.

In one or more embodiments as exemplified in FIG. 3, the PPG/electrophysiological signal processing pipeline 30 may comprise:
a collection stage 20 comprising a sensor configured to collect at least one electrophysiological signal S, e.g. a “raw” PPG signal S detected from a PPG sensor 20 provided on the vehicle steerer SW, as discussed in the foregoing;
a pre-processing stage 32 coupled to the collection stage 20 and configured to receive the collected signal S therefrom, the pre-processing stage 32 configured to apply segmenting processing to the received signal S, providing a set of filtered signals WF as a result, for instance a set of “clean” PPG waveforms comprising the signal and its derivatives obtained as a function of detected peaks and valleys in the signal, in a way per se known,
an artificial neural network (briefly, ANN) processing stage 34, coupled to the pre-processing stage 32 and configured to receive the set of filtered waveforms WF therefrom, the ANN processing stage 34 configured to produce or generate a first dataset of patterns P and to use them to alter the received set of waveforms WF, as discussed in the following, producing or generating at least one altered dataset Y as a result,
a classification stage 36, configured to receive a set of “real” measured waveforms or signals T1, T2, stored in a dedicated training database 35, the “real” signals representing expected values for the produced datasets P, Y to be asymptotically approximated; the classification stage 36 may be coupled to the ANN processing stage 34 and may be configured to apply classification processing 36 to the altered datasets Y produced therefrom, producing a classification signal T as a result; in one or more embodiments, the signal T may be assigned a logic value as a function of a comparison with a given threshold, triggering display of a label message indicative of physiological conditions of the driver D, for instance an estimate of driver's age (e.g., above or below 25 years old) or physiological state (e.g., discriminating between an awake or drowsy state).

In one or more embodiments, the system 100 may comprise a stage of measuring and pre-processing a set of PPG signals from various subjects (for instance, including the car driver D), storing such measured and pre-processed datasets as training or calibration datasets X in one or more databases 35, to be used for preliminary system calibration and/or for real time continuous update of ANN processing, as discussed in the following.

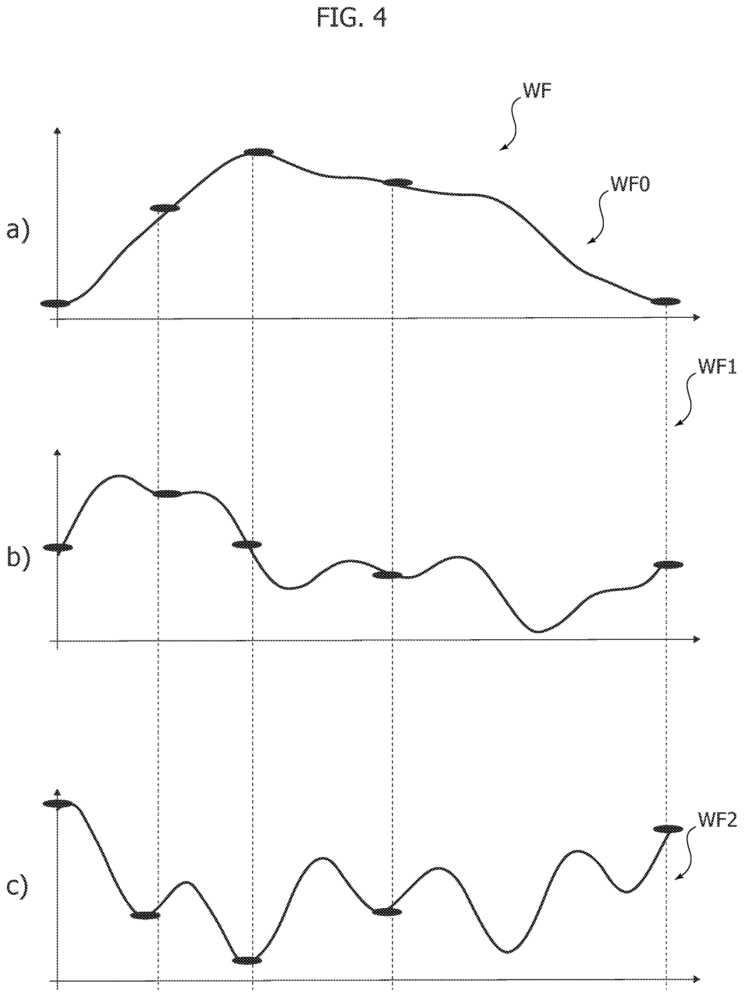

FIG. 4 is exemplary of possible time-diagrams of waveforms WF which may be provided as a result of applying filtering processing 32 to the sampled “raw” signal S.

In one more embodiment as exemplified herein, a method as discussed in Italian patent application Nr. 102017000081018 by the same Applicant may be suitable for use in the pre-processing stage 32.

In one or more embodiments, the pre-processing stage 32 may be implemented in a processing circuit of a SPC58 Chorus microcontroller unit (MCU) fabricated at STMicroelectronics.

In one or more embodiments, the set of waveforms WF may be segmented as a function of valleys and peaks detected in the PPG signal S.

For instance, the set of waveforms WF may comprise:

a segmented PPG waveform WF0, as exemplified in portion a) of FIG. 3,

a first derivative of the segmented PPG waveform WF1, as exemplified in portion b) FIG. 3,

a second derivative of the segmented waveform WF2, as exemplified in portion c) of FIG. 3.

Optionally, pre-processing may comprise normalization of waveform values in the range [0, 1].

In one or more embodiments, the set of waveforms WF (for instance comprising samples of the PPG waveform WF0, the first derivative WF1 and the second derivative WF2) may be arranged as columns/rows of a matrix which may be processed as a bi-dimensional image having waveforms sample values as pixel values.

In one or more embodiments, the matrix WF may be treated as if comprising (e.g., grayscale) pixel values, facilitating exploiting image processing techniques to process the PPG signal.

In one or more embodiments, the classification processing stage 36 may comprise a convolutional neural network, briefly CNN, processing stage, which may be trained using one or more training datasets X stored in a storage area 35 of the processing circuit 30 of the system 100.

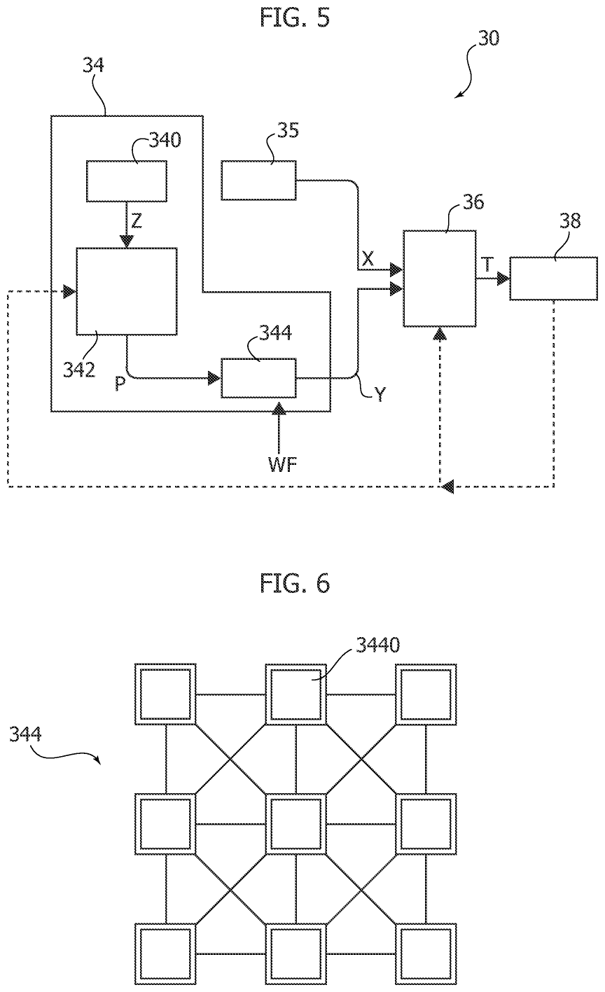

In one or more embodiments as exemplified in FIG. 5, the ANN processing stage 34 may comprise a set of processing sub-stages 340, 342, 344, as discussed in the following.

It is noted that while such stages 34, 36 and sub-stages 240, 342, 344, are discussed as separate stages in the following, in one or more embodiments they may be all incorporated, e.g. in one ANN processing stage performing all the operations of the sub-stages.

In one or more embodiments, the artificial neural network processing stage 34 may comprise:
a random noise generator stage 340, configured to provide a set of randomly generated or noise values Z, e.g. obtained by drawing a given number of values from a normal or Gaussian distribution; for instance, the a set of random values Z may be arranged as entries (e.g., columns/rows) of a matrix which may be processed as a bi-dimensional cloning template image having randomly generated sample values as pixel,
a first ANN processing stage 342, e.g., a non-linear CNN (briefly NL-CNN) cloning template stage, configured to take in the cloning template matrix comprising generated random noise (pixel) values Z and return a first dataset P, wherein such a first dataset P may comprise, for instance, an matrix of data (e.g., an image), comprising a set of possible patterns of electrophysiological signal waveforms comprising “cloned” or mimicked features which may be found in measured electrophysiological signal waveforms;
a second ANN processing stage 344, e.g., a Bio-Inspired NL-CNNs generator stage, configured to receive the first dataset P from the first ANN processing stage 342 together with the segmented set of waveforms WF and to alter the latter as a function of the first, for instance applying a distortion to the segmented set of electrophysiological waveforms WF as a function of the noise-generated dataset P provided by the first ANN stage 342, producing a second dataset comprising a set of altered electrophysiological signal waveforms F.

In one or more embodiments, the first ANN stage 342 and the classification stage 36 may resemble a generative adversarial network architecture, briefly GAN, 342, 36 wherein the ANN stage 34 may be viewed as a tailored “generator” network 340, 342, 344 and the classification stage 36 as a tailored “discrimitator” stage of the GAN.

In such a GAN 342, 36, the ANN stage may be indicated as “generator network” 342 configured to produce random-noise data and produce (new) datasets or data patterns P, Y therefrom, while the classifier stage 36 may be indicated as a “discriminator network” 36 configured to evaluate probability of whether received datasets Y from the generator network 342 matches with expected dataset values X from the calibration or training dataset or not, in a way “grading” the mimicking ability of the generator network 34 to produce datasets which resemble “real” data.

In one or more embodiments, the first ANN stage 342 may be modeled as a sort of “inverted” convolutional neural network (briefly, CNN): while applying CNN processing may comprise receiving an image and applying down-sampling thereto in order to produce a likelihood probability, the generator stage 340, 342 may take (a vector/matrix or image-grid of) random data and up-sample it. In other words, while CNNs “throws away” data through down-sampling techniques like (max-)pooling, the generator network 342 produces or processes newly drawn data samples.

In one or more embodiments, the second dataset Y may be fed to the classification or discriminator stage 36, for instance alongside the data X taken from the training datasets 35, representing a “ground-truth” dataset of expected values.

In one or more embodiments, the discriminator 36 receives “distorted” images Y and returns probabilities, that is a number between “0” and “1”, that the received dataset belongs to the training dataset X or not.

Such probabilities which represent an estimation by the discriminator, which may not necessarily be correct.

In one or more embodiments, the discriminator CNN network 36 is a convolutional network that can categorize the datasets Y provided thereto, for instance a binomial classifier labeling images as “real” or “fake”.

In one or more embodiments, for instance during a calibration or training phase, the classification signal T output by the classification stage 36 may be provided to either one or both of the generator 342 or discriminator 36 stages, forming a feedback loop therebetween, in order to fine tune values of respective ANN parameters, wherein the generator “learns” to improve the number of generated “fake” signals that are labeled as “real”, while the discriminator is “trained” to strive to achieve the opposite result, namely to avoid mislabeling “fake” data.

In one or more embodiments, the discriminator 36 may be in a feedback loop with the training dataset 35, while the first ANN 342 may be in a feedback loop with the discriminator 36.

In one or more embodiments, both the first 340, 342 and the second 344 ANN processing stages may be configured to reduce different and opposing objective functions, or loss function, in a way similar to a zero-sum game.

For the sake of simplicity, in order to provide a mathematical expression of such a relation:
the ANN stage 342 may be modeled as a non-linear function G which, when applied to the input random-noise data Z and to the segmented waveforms WF provides the altered dataset Y as a result, wherein the altered dataset may be expressed as: Y=G(Z),
the classifier stage 36 may be modeled as a non-linear function D to be applied to the input data X, Y, providing the classification signal T as a result, wherein the classification signal T may be expressed as: T=D(X, Y),
the calibration phase may use a function of training the GAN 34, 36 which may be expressed as:

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method including operations of:
segmenting at least one electrophysiological signal to produce a set of sampled waveforms;
producing a set of randomly generated noise samples;
applying artificial neural network processing to the set of sampled waveforms and to the set of randomly generated noise samples to produce at least one altered data pattern, the altered data pattern comprising the set of sampled waveforms altered as a function of the randomly generated noise samples;
providing calibration data comprising expected waveforms for the sampled waveforms in the set of sampled waveforms;
applying classifier processing to the produced at least one altered data pattern to detect a degree of resemblance between the produced at least one altered data pattern and the calibration data, the classifier processing producing classification signals having values above or below at least one threshold value as a function of the detected degree of resemblance; and
triggering a user circuit as a function of the classification signals.

2. The method of claim 1, wherein the at least one electrophysiological signal comprises at least one photopletysmography (PPG) signal.

3. The method of claim 1, wherein:
the at least one electrophysiological signal is collected from a driver of a vehicle; and
the user circuit is on board the vehicle.

4. The method of claim 3, wherein the at least one electrophysiological signal is collected from the driver of the vehicle via a PPG sensor placed on board a steering wheel of the vehicle.

5. The method of claim 1, wherein the operation of applying artificial neural network processing comprises:
applying a first artificial neural network processing to the set of randomly generated noise samples and providing a first set of generated data patterns; and
applying a second artificial neural network processing to the first set of generated data patterns and to the set of sampled waveforms and providing the at least one altered data pattern;
the second artificial neural network processing being configured to provide the at least one altered data pattern as a result of altering the set of sampled waveforms as a function of the first set of generated data patterns.

6. The method of claim 5, wherein the operation of applying the second artificial neural network processing comprises using a non-linear cellular neural network (NL-CNN) circuit.

7. The method of claim 5, wherein:
the operation of applying classifier processing comprises using convolutional neural network (CNN) processing; and
the operation of applying the first artificial neural network processing comprises using an inverse convolutional neural network processing.

8. The method of claim 1, comprising at least one of:
using the calibration data and the classification signals to configure the classifier processing to reduce occurrences of the classification signals having values indicative of a detected degree of resemblance; or
using the classification signals to configure the artificial neural network processing to increase occurrences of the classification signals having values indicative of the detected degree of resemblance.

9. A system comprising:
processing circuitry configured to perform operations of:
receiving at least one electrophysiological signal;
segmenting the at least one electrophysiological signal to produce a set of sampled waveforms;
producing a set of randomly generated noise samples;
applying artificial neural network processing to the set of sampled waveforms and to the set of randomly generated noise samples to produce at least one altered data pattern, wherein the altered data pattern comprises the set of sampled waveforms altered as a function of the randomly generated noise samples;
providing calibration data comprising expected waveforms for the sampled waveforms in the set of sampled waveforms;
applying classifier processing to the produced at least one altered data pattern to detect a degree of resemblance between the produced at least one altered data pattern and the calibration data, wherein the classifier processing produces classification signals having values above or below at least one threshold value as a function of the detected degree of resemblance; and
triggering a user circuit as a function of the classification signal.

10. The system of claim 9, wherein the system is a vehicle and further comprises:
at least one electrophysiological signal sensor configured to collect the at least one electrophysiological signal from a driver of the vehicle.

11. The system of claim 10, wherein the at least one electrophysiological signal sensor is at least one photopletismography (PPG) sensor, and the at least one electrophysiological signal is at least one PPG signal.

12. The system of claim 11, further comprising at least one driver assistance device configured to be triggered as a function of the classification signals.

13. The system of claim 9, wherein the operation of applying artificial neural network processing comprises:
applying a first artificial neural network processing to the set of randomly generated noise samples and providing a first set of generated data patterns; and
applying a second artificial neural network processing to the first set of generated data patterns and to the set of sampled waveforms and providing the at least one altered data pattern;
wherein the second artificial neural network processing is configured to provide the at least one altered data pattern as a result of altering the set of sampled waveforms as a function of the first set of generated data patterns.

14. The system of claim 13, wherein the operation of applying the second artificial neural network processing comprises using a non-linear cellular neural network (NL-CNN) circuit.

15. The system of claim 13, wherein:
the operation of applying classifier processing comprises using convolutional neural network (CNN) processing; and
the operation of applying the first artificial neural network processing comprises using an inverse convolutional neural network processing.

16. The system of claim 9, wherein the processing circuitry is configured to perform at least one of:
using the calibration data and the classification signals to configure the classifier processing to reduce occurrences of the classification signals having values indicative of a detected degree of resemblance; or
using the classification signals to configure the artificial neural network processing to increase occurrences of the classification signals having values indicative of the detected degree of resemblance.

17. A computer program product loadable into a memory of at least one processing circuit, and comprising software code portions for executing, when the product is run on the at least one processing circuit, steps of:
segmenting at least one electrophysiological signal to produce a set of sampled waveforms;
producing a set of randomly generated noise samples;
applying artificial neural network processing to the set of sampled waveforms and to the set of randomly generated noise samples to produce at least one altered data pattern, the altered data pattern comprising the set of sampled waveforms altered as a function of the randomly generated noise samples;
providing calibration data comprising expected waveforms for the sampled waveforms in the set of sampled waveforms;
applying classifier processing to the produced at least one altered data pattern to detect a degree of resemblance between the produced at least one altered data pattern and the calibration data, wherein the classifier processing produces classification signals having values above or below at least one threshold value as a function of the detected degree of resemblance; and
triggering a user circuit as a function of the classification signals.

18. The computer program product of claim 17, wherein the at least one electrophysiological signal comprises at least one photopletysmography (PPG) signal.

19. The computer program product of claim 17, wherein:
the at least one electrophysiological signal is collected from a driver of a vehicle; and
the user circuit is on board the vehicle.

20. The computer program product of claim 19, wherein the at least one electrophysiological signal is collected from the driver of the vehicle via a PPG sensor placed on board a steering wheel of the vehicle.

21. The computer program product of claim 17, wherein the step of applying artificial neural network processing comprises:
applying a first artificial neural network processing to the set of randomly generated noise samples and providing a first set of generated data patterns; and
applying a second artificial neural network processing to the first set of generated data patterns and to the set of sampled waveforms and providing the at least one altered data pattern;
wherein the second artificial neural network processing is configured to provide the at least one altered data pattern as a result of altering the set of sampled waveforms as a function of the first set of generated data patterns.

22. The computer program product of claim 17, further comprising software code portions for executing, when the product is run on the at least one processing circuit, at least one step of:
using the calibration data and the classification signals to configure the classifier processing to reduce occurrences of the classification signals having values indicative of a detected degree of resemblance; or
using the classification signals to configure the artificial neural network processing to increase occurrences of the classification signals having values indicative of the detected degree of resemblance.
